(19) United States
(12) Patent Application Publication
(10) Pub. No.: US 2005/0169948 A1
(43) Pub. Date: Aug. 4, 2005

(54) Method and compositions for treatment of acne vulgaris and acne rosacea

(76) Inventor: Joel E. Bernstein, Deerfield, IL (US)

Correspondence Address: JONES DAY, 77 WEST WACKER, CHICAGO, IL, 60601-1692, US

(21) Appl. No.: 10/767,106

(22) Filed: Jan. 29, 2004

(51) Int. Cl. A61K 31/455; A61K 31/7048; A61K 31/203; A61K 31/19; A61K 31/60

(52) U.S. Cl. 424/401; 514/355; 514/29; 514/559; 514/159; 514/574; 514/568

(57) ABSTRACT

A topical composition for the treatment of acne vulgaris or acne rosacea comprises an amount of nicotinamide and less than 1% by weight of nicotinic acid. The composition is more effective in the treatment of acne than the same composition would be without the nicotinic acid. A method of treating acne vulgaris or acne rosacea comprises the topical application of a composition comprising nicotinamide and less than 1% by weight of nicotinic acid.

Inflammatory Acne Vulgaris is a common inflammatory disease of the sebaceous glands characterized by papular, pustular and sometimes nodular or cystic cutaneous lesions. These inflammatory acne lesions are cosmetically unsightly and can in certain instances lead to permanent scarring. Acne rosacea is a condition which shares some visual similarities to acne vulgaris, but is thought to be an unrelated disease. Acne rosacea generally occurs in individuals much older than those afflicted with acne vulgaris, and is characterized by acneform lesions as well as a cutaneous vascular component marked by facial flushing and telangiectasia.

There are a variety of methods for treating inflammatory acne vulgaris including topical and systemic antibiotics and retinoids. Over twenty years ago, I patented a novel method and compositions for treating acne vulgaris (U.S. Pat. No. 4,505,896) utilizing as the active ingredients in topical formulations nicotinamide and nicotinic acid, the principal forms of vitamin B3 found in all multivitamins.

Nicotinic acid and nicotinamide are water-soluble vitamins, whose physiological active forms, nicotinamide adenine dinucleotide (NAD) and nicotinamide adenine dinucleotide phosphate (NDAP), serve a vital role as coenzymes in a variety of important metabolic reactions. Nicotinic acid is an essential dietary constituent, the lack of which leads to pellagra, a condition characterized by a erythematous skin eruption as well as gastrointestinal and neurological symptoms. Nicotinic acid and nicotinamide have been used routinely to treat pellagra for which they are therapeutic.

Clinical studies with nicotinamide involving over one thousand acne patients performed by myself or by colleagues under my supervision over the last twenty years have demonstrated that topical nicotinamide is modestly effective when compared to placebo in treating acne vulgaris or acne rosacea. However, I have found that nicotinic acid is too irritating to be applied topically on a continuous or regular basis at the concentrations (1.0% to 10%) claimed in my U.S. Pat. No. 4,505,896 patent

In searching for a method to increase the efficacy of topical nicotinamide, I discovered, surprisingly, that by adding a very small amount of nicotinic acid to topical formulations of nicotinamide, the resulting product was far more effective at treating acne vulgaris or acne rosacea than were formulations containing nicotinamide alone without increasing the irritancy potential of the formulation. I further found that formulations containing nicotinamide with small amounts of nicotinic acid could be combined with other known chemical agents known to be effective in treating acne and such resulting formulations would be more effective at treating acne than would be expected by treatment with the individual agents themselves. Such formulations include combinations of nicotinamide with small amounts of nicotinic acid and clindamycin, erythromycin, retinoic acid, benzoyl peroxide, salicylic acid, azelaic acid, tazarotene, metronidazole and other chemical agents known to be beneficial either to acne vulgaris or acne rosacea.

### DETAILED DESCRIPTION OF THE PREFERRED EMBODIMENTS

The present invention provides an improved method of and compositions for the treatment of acne vulgaris or acne rosacea involving regular applications of an effective amount of nicotinamide combined with a very small amount of nicotinic acid which enhances the activity of the nicotinamide without increasing the irritancy potential of the formulation, and rendered the combination more effective than the nicotinamide alone.

In accordance with the invention, formulations are provided that incorporate nicotinamide in concentrations from about 1-12%, as well as nicotinic acid in concentrations from about 0.005% to less than 1% in pharmaceutically acceptable vehicles for use in man and animals. A preferred concentration range for the nicotinamide is about 2-10%, and a most preferred range is about 3-7%. A preferred concentration range for the nicotinic acid is about 0.005-0.7%, and a most preferred range is about 0.05-0.2%. All concentrations are given as weight percent of a formulation.

Such formulations are designed for application to the skin and include solutions, lotions, creams, ointments, gels or pastes. Further, to the above mentioned formulations, other chemical agents known to be effective in treating acne vulgaris can be added to provide formulations that are more effective at treating acne than would be expected by treatment with the individual agents themselves. Applications of such compositions are made to the face of acne patients 1 to 4 times daily with consequent clearing or amelioration of acne vulgaris or acne rosacea skin lesions without the skin irritation commonly associated with nicotinic acid. The following examples illustrate the present invention.

### EXAMPLE 1

A 34 year-old male with inflammatory acne vulgaris applied a gel containing 4% nicotinamide and 0.1% nicotinic acid. After four weeks of treatment his face was clear of any inflammatory acne lesions.

### EXAMPLE 2

A 59 year-old female with acne rosacea applied an acne cream containing 6% nicotinamide and 0.05% nicotinic acid two times daily for two months. By the end of the treatment period her face appeared totally normal and free of rosacea lesions.

### EXAMPLE 3

A 29 year-old female with acne vulgaris and more than 10 inflammatory acne papules or pustules applied an emulsion containing 4% nicotinamide and 0.05% nicotinic acid. Although she had used a similar emulsion containing 4% nicotinamide without the nicotinic acid, and had not been responsive to it, with the new emulsion containing both nicotinamide and nicotinic acid the patient's acne lesions cleared completely after four weeks of once daily treatment.

1. A method of treating acne vulgaris and acne rosacea comprising applying to the skin of patients afflicted with such disorders a topical composition comprising an amount of nicotinamide combined with less than 1% by weight of nicotinic acid, the resulting formulation being more effective than said amount of nicotinamide by itself.

2. The method of claim 1 wherein said composition comprises about 1-12% nicotinamide by weight.

3. The method of claim 2 wherein said composition comprises about 2-10% nicotinamide by weight and about 0.005-0.7% nicotinic acid by weight.

4. The method of claim 3 wherein said composition comprises about 3-7% nicotinamide by weight and about 0.05-0.2% nicotinic acid by weight.

5. A method of treating acne vulgaris and acne rosacea comprising adding to the composition of claim 1 other known acne treatment agents.

6. The method of claim 5 wherein said other known acne treatment agents are selected from the group consisting of clindamycin, erythromycin, retinoic acid, benzoyl peroxide, salicylic acid, azelaic acid, tazarotene, and metronidazole.

7. A topical composition effective for treating acne vulgaris and acne rosacea comprising a pharmaceutically acceptable carrier and an amount of nicotinamide in combination with less than 1% by weight of nicotinic acid, the resulting formulation being more effective than said amount of nicotinamide by itself.

8. The composition of claim 7 wherein said composition comprises about 1-12% nicotinamide by weight.

9. The composition of claim 8 wherein said composition comprises about 2-10% nicotinamide by weight and about 0.005-0.7% nicotinic acid by weight.

10. The composition of claim 9 wherein said composition comprises about 3-7% nicotinamide by weight and about 0.05-0.2% nicotinic acid by weight.

11. The composition of claim 7 wherein other known acne treatment agents are included in the composition.

12. The composition of claim 9 wherein the other known acne treatment agents are selected from the group consisting of clindamycin, erythromycin, retinoic acid, benzoyl peroxide, salicylic acid, azelaic acid, tazarotene, and metronidazole.
